(19) United States
(12) Patent Application Publication
(10) Pub. No.: US 2023/0190142 A1
(43) Pub. Date: Jun. 22, 2023

(54) SYSTEM AND METHOD OF TESTING A HEARING FUNCTION

(71) Applicant: Children's Hearing Foundation, Taipei (TW)

(72) Inventors: Pei-Hua CHEN, Taipei (TW); Chia-Ying CHU, Taipei (TW); Yu-Chen HUNG, Taipei (TW)

(21) Appl. No.: 18/081,360

(22) Filed: Dec. 14, 2022

Related U.S. Application Data
(60) Provisional application No. 63/292,126, filed on Dec. 21, 2021.

(51) Int. Cl. A61B 5/12; A61B 5/00

(52) U.S. Cl. CPC A61B 5/125; A61B 5/4803

(57) ABSTRACT

A system and method of adaptively testing a hearing function performs a hearing test including outputting a sound of a tested word chosen from a selected one of the word lists, receiving an input of a patient in response to the outputted sound, and determining whether the input matches with the tested word. The hearing test is repeated until a testing end condition is satisfied, wherein the selected one of the word lists in a next iteration of the hearing test has a next higher level of difficulty when the input matches with the tested word in a previous iteration of the hearing test, and the selected one of the word lists in the next iteration of the hearing test has a next lower level of difficulty when the input does not match with the tested word in the previous iteration of the hearing test.

### CROSS REFERENCE TO RELATED APPLICATION(S)

This application claims priority to U.S. Provisional Pat. Application No. 63/292,126 filed on Dec. 21, 2021, the disclosure of which is hereby incorporated by reference.

### BACKGROUND

### 1. Field of the Invention

The present invention relates to systems and methods of testing a hearing function, in particular to systems and methods that can swiftly provide accurate hearing test results.

### 2. Description of the Related Art

A speech discrimination score is typically a hearing test that can be conducted by an audiologist to evaluate a patient’s ability to hear and identify the sound of words. To ensure the accuracy of the test results, the patient is usually tested with a predetermined number of words (typically 25 to 50 words), and the hearing function of the patient is evaluated after all the words are tested.

Because the patient is tested with a significant number of words, the conventional testing method requires the patient’s attention for a long period of time. As a result, the conventional testing method may not be adapted for young children who are easily distracted and cannot stay focused. When adults or young children take the test to screen for hearing loss, fatigue may occur as the test requires that the patient stays highly focused for a long period of time, which would affect the patient’s response. Continuously testing numerous words may also induce memory effects in the patient and consequently alter the accuracy of the test. In addition, the requisite amount of time for completing the conventional testing method may be a factor limiting its implementation.

Therefore, there is a need for an improved method of testing a hearing function that can provide accurate test results in a more efficient manner and address at least the foregoing issues.

### SUMMARY

The present application describes a system and method of adaptively testing a hearing function. The method is less-time consuming as accurate test results can be obtained with a reduced number of test questions, and can prevent memory-induced distortion in the test results.

According to an embodiment, a hearing testing system includes a user interface device, a data storage device, and a processing unit connected to the user interface device and the data storage device. The data storage device is configured to store a database including a plurality of word lists, each of the word lists including a predetermined number of words to test, and the word lists being respectively associated with different levels of difficulty. The processing unit is configured to execute a program code that causes the hearing testing system to: perform a hearing test including outputting a sound of a tested word chosen from a selected one of the word lists, receiving an input of a patient in response to the outputted sound, and determining whether the input matches with the tested word; repeat the hearing test until a testing end condition is satisfied, wherein the selected one of the word lists in a next iteration of the hearing test has a next higher level of difficulty when the input matches with the tested word in a previous iteration of the hearing test, and the selected one of the word lists in the next iteration of the hearing test has a next lower level of difficulty when the input does not match with the tested word in the previous iteration of the hearing test; and when the testing end condition is satisfied, stop the hearing test and output a test result.

Moreover, the present application describes a computer-implemented method of testing a hearing function. According to an embodiment, the method includes: storing a plurality of word lists, each of the word lists including a predetermined number of words to test, and the word lists being respectively associated with different levels of difficulty; performing a hearing test including outputting a sound of a tested word chosen from a selected one of the word lists, receiving an input of a patient in response to the outputted sound, and determining whether the input matches with the tested word; repeating the hearing test until a testing end condition is satisfied, wherein the selected one of the word lists in a next iteration of the hearing test has a next higher level of difficulty when the input matches with the tested word in a previous iteration of the hearing test, and the selected one of the word lists in the next iteration of the hearing test has a next lower level of difficulty when the input does not match with the tested word in the previous iteration of the hearing test; and when the testing end condition is satisfied, stopping the hearing test and outputting a test result.

### BRIEF DESCRIPTION OF THE DRAWINGS

FIG. 1 is a flowchart illustrating an embodiment of a hearing testing method;

FIG. 2 is a block diagram illustrating an embodiment of a hearing testing system; and

FIG. 3 is a schematic diagram illustrating an example of application of the hearing testing method.

### DETAILED DESCRIPTION OF THE EMBODIMENTS

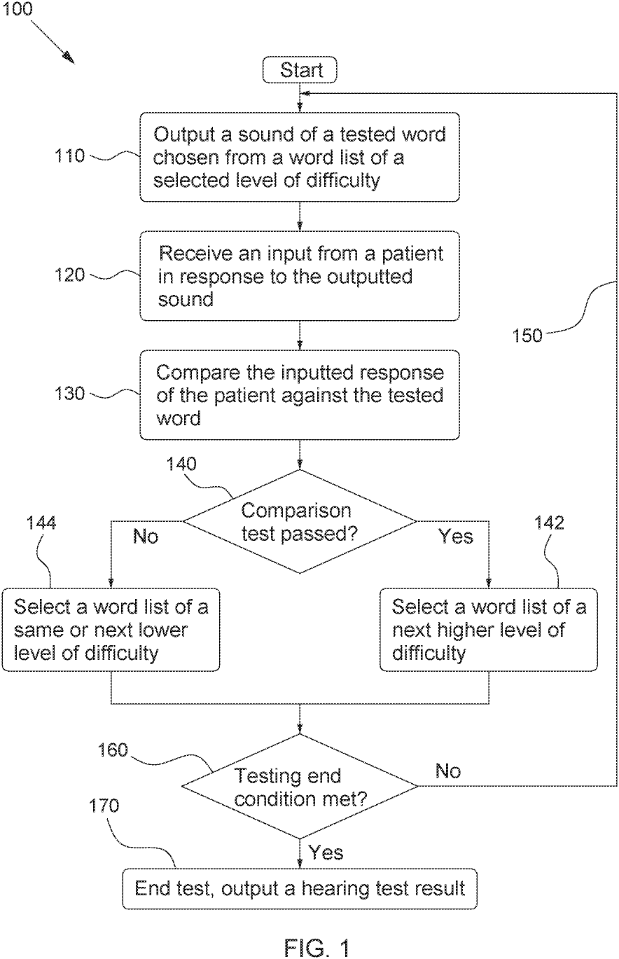

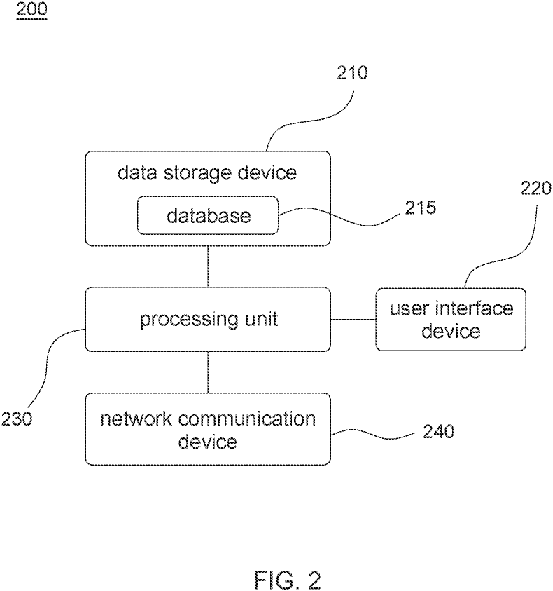

FIG. 1 is a flowchart illustrating an embodiment of a hearing testing method 100, and FIG. 2 is a block diagram illustrating an embodiment of a hearing testing system 200. Referring to FIGS. 1 and 2, the hearing testing method 100 may be conducted by a professional (e.g., an audiologist, a manufacturer and/or seller of hearing aids, etc.) to evaluate a hearing function of a patient using the hearing testing system 200. The patient may wear a hearing aid while he/she is subjected to the hearing testing method 100.

In step 110, the hearing testing system 200 can output a sound of a tested word chosen from a selected word list. The tested word can be selected from a database including multiple word lists, wherein each word list contains a predetermined number of words corresponding to a predetermined level of difficulty. For example, the database may include, without limitation, 6 or 10 word lists. Each word list may exemplarily include, without limitation, 20 to 25 words, or 26 to 50 words. Each word list can correspond to a level of difficulty, e.g., from any level from Level 1 to Level 6, or from Level 1 to Level 10. The levels of difficulty may be established based on the Item Response Theory (IRT) method. For example, the tested words may be grouped into 6 word lists having 6 different levels of difficulty. It will be appreciated, however, that the number of levels of difficulty may vary according to the actual testing needs, and the number of words associated with each level of difficulty may also be adjusted according to the actual testing needs.

In step 120, the hearing testing system 200 receives an input that is entered by the patient in response to the outputted sound of the tested word. For example, the tested word may be “tree”, and the patient can input a response to the hearing testing system 200 after hearing the outputted sound of the word “tree”.

The hearing testing system 200 in step 130 compares the inputted response of the patient against the tested word, and in next step 140 determines whether the inputted response has passed the comparison test. The inputted response passes the comparison test when the inputted response matches with the tested word, and fails to the comparison test when the inputted response does not match with the tested word.

Depending on whether the response inputted by the patient has passed or has not passed the comparison test, step 142 or 144 then may be performed to select another word to test.

According to an example, supposing that the tested word in step 110 is “tree”, and the word “tree” is arbitrarily assumed to be in a word list of difficulty Level 2. In step 120, after hearing the sound of the word “tree”, the patient can input a response into the hearing testing system 200, e.g., by using a user interface device of the hearing testing system 200 to enter the spelling or phonetic notation of “tree”. In case the comparison test in step 140 is passed, e.g., when the patient has entered the correct spelling “T-R-E-E″ or the correct phonetic notation “/tri/”, step 142 is performed whereby the hearing testing system 200 may select another word to test from a next higher level of difficulty (e.g., the word “shade” is selected, which is arbitrarily assumed to be in the word list of higher difficulty Level 3).

On the other hand, in case the comparison test in step 140 has not passed, e.g., when the patient has entered the incorrect spelling “D-R-E-E” or the incorrect phonetic notation “/dri/” in response to the outputted sound of the tested word “tree”, step 144 is performed whereby the hearing testing system 200 may select another word to test from a same or a next lower level of difficulty (e.g., the word “bee” is selected, which is arbitrarily assumed to be in the word list of lower difficulty Level 1).

In step 160, the hearing testing system 200 determines whether a testing end condition is satisfied. If the hearing testing system 200 in step 160 determines that no testing end condition is satisfied, the method loops back at 150 and the hearing test including steps 110, 120, 130 and 140 is repeatedly applied for a word of the word list selected in step 142 or 144 (e.g., the word “shade” or “bee” as mentioned previously). If the hearing testing system 200 in step 160 determines that a testing end condition is satisfied, then the hearing testing system 200 in step 170 ends the hearing test and outputs a hearing test result. Accordingly, the hearing test can be repeated by selectively varying the levels of difficulty according to the patient’s input until the hearing testing system 200 determines that a testing end condition is satisfied.

According an embodiment, the hearing test ends when one of the following conditions is satisfied:
(A) the inputted response matches with the tested word of the word list having the highest level of difficulty (e.g., Level 6) for a predetermined number of times;
(B) as the hearing test alternates between two word lists of successive levels of difficulty, the inputted response matches with the tested word for a predetermined number of times;
(C) all the words of a word list of a level of difficulty have been tested;
(D) the inputted response does not match with the tested word for a predetermined number of successive iterations of the hearing test (e.g., two, three or more successive iterations); and
(E) the inputted response does not match with the tested word selected from the word list of the lowest level of difficulty (e.g., Level 1) for a predetermined number of successive iterations of the hearing test.

Referring to FIGS. 1 and 2, the hearing testing system 200 can be used to conduct the hearing testing method 100. The hearing testing system 200 can include a data storage device 210, a user interface device 220, and a processing unit 230 connected to the data storage device 210 and the user interface device 220. Optionally, the hearing testing system 200 may further include a network communication device 240 connected to the processing unit 230, wherein the hearing testing system 200 can communicate and exchange data with an external device (not shown) through the network communication device 240.

According to an embodiment, the data storage device 210 is configured to store a database 215 including multiple word lists. Each word list includes a predetermined number of words to test and is associated with a corresponding level of difficulty. For example, each word list may include, but is not limited to, 20 to 25 words, and the level of difficulty may include, but is not limited to, any level from Level 1 to Level 6. The user interface device 220 is configured to output a sound of a tested word to the patient, and to receive an input by the patient in response to the outputted sound. The processing unit 230 is configured to communicate with the data storage device 210 and the user interface device 220, access to the data storage device 210, receive a patient’s input from the user interface device 220, and issue a command to the user interface device 220 to output sound.

The processing unit 230 is respectively connected to the data storage device 210, the user interface device 220 and the network communication device 240. According to an embodiment, the processing unit 230 is configured to execute the following: issuing an instruction to the user interface device 220 to output a sound of a tested word that is selected from the database 215, wherein the tested word is from a word list having a predetermined level of difficulty; determining whether the patient’s input in response to the outputted sound matches with the tested word; and according to the result of this determination, selecting another word to test and issuing an instruction to the user interface device 220 to output a sound of the other word to test, wherein the other word to test is selected from a word list of a next higher level of difficulty when the patient’s input matches with the previously tested word, and the other word to test is selected from a word list of a next lower level of difficulty when the patient’s input does not match with the previously tested word. The processing unit 230 can repeat the aforementioned steps until a testing end condition is satisfied. When the testing end condition is satisfied, the processing unit 230 stops executing the aforementioned steps and assesses and outputs a testing result.

The hearing testing system 200 may be a computer device, which can include, without limitation, portable devices (e.g., a tablet computer, a laptop computer, and the like) and desktop computers. The data storage device 210 may be a storage device internal to the computer device or portable device, or may be an external storage device outside the computer device or portable device (e.g., the external storage device may be at a remote location and accessed via a network connection). The user interface device 220 can include, without limitation, a display device, a touch screen, a speaker, a keyboard, a microphone, a pointing device, and the like. The processing unit 230 can include, without limitation, one or more processors/microprocessors, and a memory (e.g., volatile memory such as RAM) for loading a program code, wherein the program code when executed by the processing unit 230 causes the hearing testing system 200 to perform the steps of the hearing testing method 100 described herein. Optionally, the user interface device 220 may be an external device (e.g., a separate tablet computer) independent of the processing unit 230, or a peripheral device of the processing unit 230.

In some embodiments, the hearing testing system 200 may also be implemented into a system of networked computers, wherein the processing unit 230 may be provided in a server computer, and the user interface device 220 may be a terminal device remotely connected to the processing unit 230.

The words to test contained in the database 215 can include monosyllabic words or multisyllabic words. For example, with respect to Chinese language, the database 215 can include single-character words corresponding to monosyllabic words, or multi-character words corresponding to multisyllabic words. The words to test may be classified into word lists of different levels of difficulty based on the IRT.

A patient can input a response by using a word input device (e.g., a keyboard, a touch screen), a voice input device (e.g., a microphone) or other devices (e.g., a pointing device). For example, the patient can use the user interface device 220 to input a response through one of the following methods: inputting a phonetic notation (e.g., based on the Zhuyin or Pinyi phonetic alphabet for Chinese language) that the patient believes to correspond to the tested word, inputting or selecting a graphics that the patient believes to correspond to the tested word, and inputting a sound that the patient believes to correspond to the tested word. For illustration, supposing that the hearing testing system 200 outputs, e.g., the sound of the tested word “tree” (which may exemplarily correspond to difficulty Level 2 as described previously). A patient can use a word input device (e.g., a keyboard or a touch screen) to input a response that the patient believes to correspond to the tested word “tree” (e.g., by entering the spelling “T-R-E-E″ or the phonetic notation “/tri/”), can use a voice input device (e.g., a microphone) to input a sound that the patient believes to correspond to the tested word “tree”, or can use a pointing device to select a graphics or photo on a display screen that the patient believes to correspond to the tested word “tree”.

As previously described, the processing unit 230 is configured to determine whether the response inputted by the patient matches with the tested word, and select a following word to test from a higher level of difficulty (e.g., Level 3) or a lower level of difficulty (e.g., Level 1) depending on whether the response matches or does not match the previously tested word. Like previously described, the hearing testing system 200 can repeatedly apply the hearing test, and end the hearing test when one of the preset testing end conditions is satisfied.

As previously described, all of the words contained in the database 215 can be exemplarily classified into 6 word lists of different levels of difficulty based on the IRT, each word list including a predetermined number of words (e.g., 20 to 25 words). The word list of Level 6 can be defined as having the highest level of difficulty, and the word list of Level 1 can be defined as having the lowest level of difficulty. According to an example, the classification into multiple word lists of different levels of difficulty can include the following steps: testing all of the words on a group of adults having a normal hearing function, and recording the results of the responses as “pass” or “fail”; according to the results of the responses, calculating a word difficulty level; and grouping the words into 6 levels of difficulty according to difficulty grades expressed in logits that are computed for the tested words. Table 1 below illustrates an example of word grouping into 6 levels of difficulty:

TABLE 1

| | Level 1 | Level 2 | Level 3 | Level 4 | Level 5 | Level 6 |
|---|---|---|---|---|---|---|
| Number of words | 4 | 11 | 11 | 11 | 8 | 5 |
| Average grade (logits) | -3.33 | -1.57 | -0.37 | 0.38 | 1.35 | 2.35 |
| Range (logits) | -3.95~-2.70 | -1.93~-1.05 | -0.80~-0.12 | 0.24-0.73 | 1.05-1.88 | 2.08-2.53 |

According to an embodiment implemented for Chinese language, a test material applied in a conventional Mandarin Monosyllable Recognition Test (MMRT) may be used as a basis for the hearing testing method 100. All of the words subjected to the MMRT are classified into multiple word lists of different levels of difficulty based on the IRT as described previously. Then the word lists can be applied in the hearing testing system and method described herein. The hearing testing method 100 described herein may also be referred to as an Adaptive Mandarin Monosyllable Recognition Test (AMMRT) or adaptive hearing testing method.

A statistical test may be conducted to determine whether the AMMRT method provides test results that are consistent with those of the MMRT method. For example, each of the AMMRT method and the MMRT method is respectively conducted on a pool of 37 adults, and a conventional chi-square test is applied to determine whether the test results of the AMMRT method are consistent with those of the MMRT method, the test results of the MMRT method being used as reference. In the chi-square test, a p-value that is less than the significance level of 0.05 indicates that there is consistency between the AMMRT method and the MMRT method. The results of the chi-square test is illustrated in Table 2 below.

TABLE 2

Sample n = 37

| | AMMRT | | χ² | df | p |
|---|---|---|---|---|---|
| MMRT | Fail | Pass | | | |
| Fail | 3 | 10 | 6.03 | 1 | 0.014 |
| Pass | 0 | 24 | | | |

The results obtained in the chi-square test are χ²=6.03 and p=0.014 for a degree of freedom df equal to 1, wherein the p-value of 0.014 indicates that the test results obtained with the AMMRT method and the test results obtained with the MMRT method are statistically consistent. In other words, an individual who passes the conventional MMRT method will also pass the AMMRT method. Accordingly, the conventional MMRT method can be replaced with the adaptive hearing testing method described herein, which is less time-consuming and can provide test results in a more efficient manner.

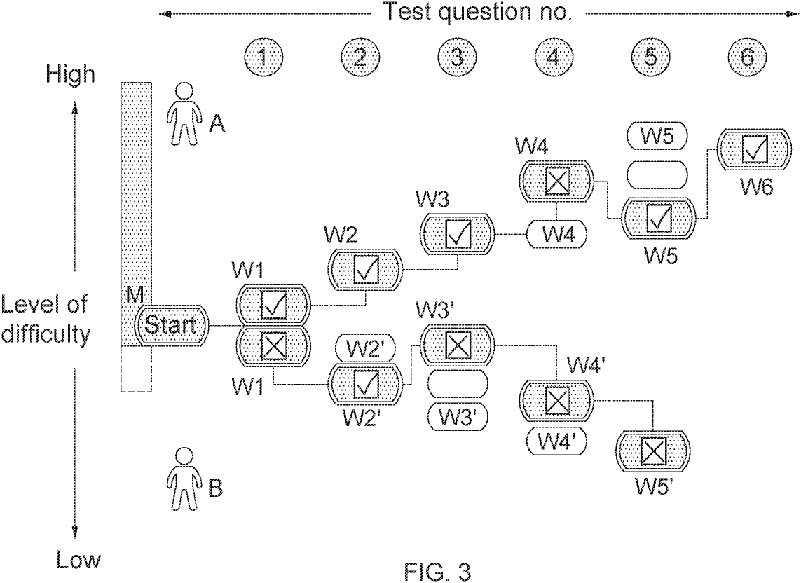

FIG. 3 is a schematic diagram illustrating an example of application of the hearing testing method 100, which can be respectively conducted for each of a patient A and a patient B by using the hearing testing system 200. In the example of FIG. 3, the hearing testing method has 6 levels of difficulty. For each of the patient A and B, the hearing testing method 100 can start by providing a word W1 selected from a word list of a same intermediate level of difficulty to the patient.

With respect to the patient A, supposing that the initial response is correct and matches with the tested word W1 (which is marked with the symbol V). The hearing testing system 200 then provides a word W2 of a next higher level of difficulty (i.e., higher than the level of difficulty of the previously tested word W1) to the patient A, and the patient A also inputs a correct response to the tested word W2. Subsequently, the patient A also passes the hearing test for a word W3 of a next higher level of difficulty (i.e., higher than the level of difficulty of the previously tested word W2), but fails to input a correct response for a following word W4 having a level of difficulty higher than the level of difficulty of the previously tested word W3 (which is marked with the symbol X). As a result, the hearing testing system 200 then selects and tests a word W5 of a level of difficulty lower than the level of difficulty of the previously tested word W4, and then can raise the level of difficult as the patient A inputs a correct response. Eventually, the patient A inputs a correct response to a word W6 of the highest level of difficulty. Supposing that the aforementioned testing end condition (A) is exemplarily defined as the occurrence of an inputted response matching once with the tested word of the word list having the highest level of difficulty, the input of the correct response to the word W6 of the highest level of difficulty thus satisfies the testing end condition (A). Accordingly, the hearing test ends, and the hearing testing system 200 can output a test result indicating that the patient A has passed the hearing test.

With respect to the patient B, supposing that the initially inputted response is incorrect and does not match with the initially tested word W1 (which is marked with the symbol X). The hearing testing system 200 then selects and provides a word W2′ of a next lower level of difficulty (i.e., lower than the level of difficulty of the previously tested word W1) to the patient B, and the patient B at this time inputs a correct response to the tested word W2′. Subsequently, the hearing testing system 200 selects and provides a word W3′ of a level of difficulty higher than the level of difficulty of the previously tested word W2′, but the patient B fails to input a correct response for the tested word W3′ as well as for the following tested words W4′ and W5′ of increasingly lower levels of difficulty. Supposing that the aforementioned testing end condition (D) is exemplarily defined as the occurrence of three successive failed hearing tests, the incorrect responses inputted by the patient B for the tested words W3′, W4′ and W5′ satisfy the testing end condition (D). Accordingly, the hearing test ends, and the hearing testing system 200 outputs a testing result indicating that the patient B has not passed the hearing test.

Based on the aforementioned examples, the patient A can obtain the hearing test result after the completion of 6 test questions, and the patient B can obtain the hearing test result after the completion of 5 test questions. In case a patient alternately provides correct and incorrect responses for tested words of a same level of difficulty (e.g., for the tested words W2′ and W4′ shown in FIG. 3),which may increase the number of applied test questions, the total number of applied test questions required for obtaining an accurate test result is still substantially lower than the number of test questions (generally at least 25 to 50 test questions) required in the conventional testing method. Accordingly, the hearing testing method 100 is significantly less time-consuming than the conventional hearing testing method, and can provide test results that are likewise accurate and consistent to the test results of the conventional hearing testing method.

The system and method described herein can test a patient’s hearing function based on a database of homogeneous words, which can preclude distortion that may be induced by the varying skills of the patient subjected to the hearing test. The hearing test can adaptively switch between multiple word lists of different levels of difficulty so as to reduce the difficulty gap between the tested words. In addition, before the hearing testing method is fully applied, a pretest may be conducted to ensure that the patient passes the hearing test for at least the lowest level of difficulty. This allows to detect patients having insufficient skill to undergo the full hearing testing method as described herein.

Realizations of the systems and methods have been described only in the context of particular embodiments. These embodiments are meant to be illustrative and not limiting. Many variations, modifications, additions, and improvements are possible. Accordingly, plural instances may be provided for components described herein as a single instance. Structures and functionality presented as discrete components in the exemplary configurations may be implemented as a combined structure or component. These and other variations, modifications, additions, and improvements may fall within the scope of the claims that follow.

What is claimed is:

1. A hearing testing system comprising:
a user interface device;
a data storage device configured to store a database including a plurality of word lists, each of the word lists including a predetermined number of words to test, and the word lists being respectively associated with different levels of difficulty; and
a processing unit connected to the user interface device and the data storage device, wherein the processing unit is configured to execute a program code that causes the hearing testing system to:
perform a hearing test including outputting a sound of a tested word chosen from a selected one of the word lists, receiving an input of a patient in response to the outputted sound, and determining whether the input matches with the tested word;
repeat the hearing test until a testing end condition is satisfied, wherein the selected one of the word lists in a next iteration of the hearing test has a next higher level of difficulty when the input matches with the tested word in a previous iteration of the hearing test, and the selected one of the word lists in the next iteration of the hearing test has a next lower level of difficulty when the input does not match with the tested word in the previous iteration of the hearing test; and
when the testing end condition is satisfied, stop the hearing test and output a test result.

2. The hearing testing system according to claim 1, wherein the testing end condition includes one or more of the following:
the input matches with the tested word of the word list having a highest level of difficulty for a predetermined number of times;
as the hearing test alternates between two word lists of successive levels of difficulty, the input matches with the tested word for a predetermined number of times;
a word list of a level of difficulty has been entirely tested;
the input does not match with the tested word for a predetermined number of successive iterations of the hearing test; and
the input does not match with the tested word selected from the word list of a lowest level of difficulty for a predetermined number of successive iterations of the hearing test.

3. The hearing testing system according to claim according to claim 1, further comprising a network communication device.

4. The hearing testing system according to claim 1, wherein the database includes 6 word lists corresponding to 6 levels of difficulty.

5. The hearing testing system according to claim 1, wherein the database includes 10 word lists corresponding to 10 levels of difficulty.

6. The hearing testing system according to claim 1, wherein each of the word lists includes 20 to 25 words.

7. The hearing testing system according to claim 1, wherein each of the word lists includes 25 to 50 words.

8. The hearing testing system according to claim 1, wherein each of the word lists includes monosyllable words or multisyllable words.

9. The hearing testing system according to claim 1, wherein the user interface device comprises a word input/output device or a voice input/output device.

10. The hearing testing system according to claim 1, wherein the user interface device is configured to receive the input of a patient in response to the outputted sound, the input including a phonetic notation based on the Zhuyin or Pinyi phonetic alphabet, a graphics or a sound.

11. A computer-implemented method of testing a hearing function, comprising:
storing a plurality of word lists, each of the word lists including a predetermined number of words to test, and the word lists being respectively associated with different levels of difficulty;
performing a hearing test including outputting a sound of a tested word chosen from a selected one of the word lists, receiving an input of a patient in response to the outputted sound, and determining whether the input matches with the tested word;
repeating the hearing test until a testing end condition is satisfied, wherein the selected one of the word lists in a next iteration of the hearing test has a next higher level of difficulty when the input matches with the tested word in a previous iteration of the hearing test, and the selected one of the word lists in the next iteration of the hearing test has a next lower level of difficulty when the input does not match with the tested word in the previous iteration of the hearing test; and
when the testing end condition is satisfied, stopping the hearing test and outputting a test result.

12. The computer-implemented method according to claim 11, wherein the testing end condition includes one or more of the following:
the input matches with the tested word of the word list having a highest level of difficulty for a predetermined number of times;
as the hearing test alternates between two word lists of successive levels of difficulty, the input matches with the tested word for a predetermined number of times;
a word list of a level of difficulty has been entirely tested;
the input does not match with the tested word for a predetermined number of successive iterations of the hearing test; and
the input does not match with the tested word selected from the word list of a lowest level of difficulty for a predetermined number of successive iterations of the hearing test.

13. The computer-implemented method according to claim 11, wherein the input of the patient in response to the outputted sound includes a phonetic notation based on the Zhuyin or Pinyi phonetic alphabet, a graphics or a sound.
